(19) United States
(12) Patent Application Publication
(10) Pub. No.: US 2018/0193121 A1
(43) Pub. Date: Jul. 12, 2018

(54) TOOTH CUP, TOOTH BRUSH AND ORAL CARE SYSTEM

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Detao ZHAO, Beijing (CN); Yangheng LI, Beijing (CN); Pengju ZHANG, Beijing (CN); Xin LI, Beijing (CN)

(21) Appl. No.: 15/526,652

(22) PCT Filed: Aug. 15, 2016
(86) PCT No.: PCT/CN2016/095277

§ 371 Date: May 12, 2017

(30) Foreign Application Priority Data
Apr. 7, 2016 (CN) 201610213032.9

(51) Int. Cl. A61C 17/22

(52) U.S. Cl. CPC A61C 17/221; A61L 2/10; A61C 17/224

(57) ABSTRACT

There is described a tooth cup comprising a cup body in which it is integrated with: a controller configured to issue a purification command based on a predetermined condition; and a purifier configured to purify the tooth cup in response to receiving the purification command issued by the controller. In addition, it further describes a tooth brush and an oral cavity care system. This helps people with improving the oral cavity health in daily lives.

### TECHNICAL FIELD

The present disclosure relates to the field of smart life and, in particular, to a tooth cup, a tooth brush and an oral care system.

### BACKGROUND

As the life pace is growing faster, problems of oral health tend to be neglected more and more easily. Improper ways and time lengths of brushing tooth are harmful to oral health; the growing of unhealthy substances in a tooth cup, such as microorganism, can also cause oral health problems. Generally, a common tooth cup functions only to hold water and accommodate a tooth brush, and is easily adhered with unhealthy substances, and moreover, a common tooth brush may be easily adhered with unhealthy substances and has also other serious problems.

### SUMMARY

Therefore, it is desired to provide a device that helps people to improve the oral health in daily lives.

According to an aspect of the present disclosure, a tooth cup is provided, comprising a cup body in which it is integrated with: a controller configured to issue a purification command based on a predetermined condition; and a purifier configured to purify the tooth cup in response to receiving the purification command issued by the controller. This helps, for example, a user to purify the tooth cup in accordance with appropriate conditions.

Optionally, in the cup body, it is integrated with a power supply connected to the controller and the purifier. The power supply may provide the power required for the operation of the controller and/or the purifier.

Optionally, the purifier comprises a microorganism purifier. The microorganism purifier may be a drier and/or an ultraviolet sterilizer, for example. This is favorable to enhancing the purification effect.

Optionally, in the cup body, it is further integrated with a detector for detecting a degree of cleanness in the tooth cup. This can achieve monitoring and tracing the degree of cleanness in the tooth cup. In an example, the controller may be configured to issue a purification command based on such predetermined condition that the degree of cleanness detected by the detector is below a cleanness threshold. This helps to maintain a long period of relative healthiness of the environment in the tooth cup. As another example, the controller may be configured to issue a purification command based on such predetermined condition that the water volume in the tooth cup is reduced below a predetermined water volume threshold. This can enable the purification of the tooth cup in the first time, thereby largely reducing the work load required by the purification. Additionally or alternatively, the purifier may also be configured to purify the tooth cup in response to receiving a purification command issued by a user.

Optionally, in the cup body, it is further integrated with a Bluetooth module through which the controller can communicate with an external device. This is advantageous, because the controller can receive a video, a picture, time information, etc. from the external device, and thereby complete the brushing process with a higher quality; and/or, the controller can also output information of the environment in the cup to an external mobile device, in order to enable a user to trace the degree of cleanness in the tooth cup and thus to better grasp the timing of brushing his/her tooth.

Optionally, in the cup body, it is further integrated with a display for displaying data under the control of the controller. The display can, for example: display the information of a video intuitively, so as to standardize the brushing action in the process of brushing; display dynamic image information, so as to cultivate an interest of a child in brushing his/her tooth; display time information, so as to easily ensure the time for the tooth brushing, and so on.

Optionally, the detector may comprise a testing probe which comprises luciferin-luciferase thereon and may be configured to capture and detect a luminous value of a photon generated from an interaction between the luciferin-luciferase and the remaining liquid in the tooth cup. A degree of cleanness in the tooth cup may be derived from the luminous value.

According to another aspect of the present disclosure, a tooth brush is provided, comprising a brush head in which it is integrated with: a controller configured to issue a purification command based on a predetermined condition; and a purifier configured to purify the brush head in response to receiving the purification command issued by the controller. This helps, for example, a user to purify the brush head in accordance with appropriate conditions.

Optionally, the tooth brush may further be integrated with a power supply connected to the controller and the purifier. The power supply may provide the power required for the operation of the controller and/or the purifier.

Optionally, the purifier comprises a microorganism purifier. The microorganism purifier may be a drier and/or an ultraviolet sterilizer, for example. This is favorable to enhancing the purification effect.

Optionally, in the tooth brush, it is further integrated with a detector for detecting a degree of cleanness of the brush head. This can achieve monitoring and tracing the degree of cleanness of the brush head. In an example, the controller may be configured to issue a purification command based on such predetermined condition that the degree of cleanness as detected by the detector is below a cleanness threshold. This helps to maintain a long period of relative healthiness of the brush head. As another example, the controller may be configured to issue a purification command based on such predetermined condition that the water volume on the brush head is reduced below a predetermined water volume threshold. This can largely reduce the work load required by purification. Additionally or alternatively, the purifier may also be configured to purify the brush head in response to receiving a purification command issued by a user.

Optionally, in the tooth brush, it is further integrated with a Bluetooth module through which the controller can communicate with an external device. This is advantageous, because the controller can receive a video, a picture, time information, etc. from the external device, and thereby complete the brushing process with a higher quality; and/or, the controller can also output environment information of the tooth brush, such as the degree of cleanness, to an external mobile device, in order to enable a user to trace the degree of cleanness of the tooth brush and thus to better grasp the timing of brushing his/her tooth. Optionally, in the tooth brush it is also integrated with a display for displaying the above data under the control of the controller.

According to another aspect of the present disclosure, an oral cavity care system is provided, comprising any of the tooth cups and/or any of the tooth brushes as described above.

According to embodiments to be described below, these and other advantages of the present disclosure will become apparent, and will be elucidated with reference to the following described embodiments.

### DESCRIPTION OF THE DRAWINGS

Now the present disclosure will be described in more detail and with reference to the accompanying drawings, wherein:

FIG. 1 is a schematic diagram of a tooth cup according to an embodiment of the present disclosure;

FIG. 2 is an exemplary structural diagram of a cup body of the tooth cup according to an embodiment of the present disclosure.

FIG. 3 is a schematic diagram of a tooth brush according to an embodiment of the present disclosure.

### DETAILED DESCRIPTION

The following description provides specific details for a thorough understanding of, and enabling description for, various embodiments of the disclosure. One skilled in the art will understand that the technical solution of the disclosure may be practiced without many of these details. In some instances, well-known structures and functions have not been shown or described in detail to avoid unnecessarily obscuring the description of embodiments of the disclosure. It is intended that the terminology used in this disclosure be interpreted in its broadest reasonable manner, even though it is being used in conjunction with a detailed description of certain embodiments of the disclosure. For example, the terms “based on or in response to” are not exclusive, and are equivalent with the terms “at least partially based on or in response to”, and also include “based on” some additional factors that are not described in this context.

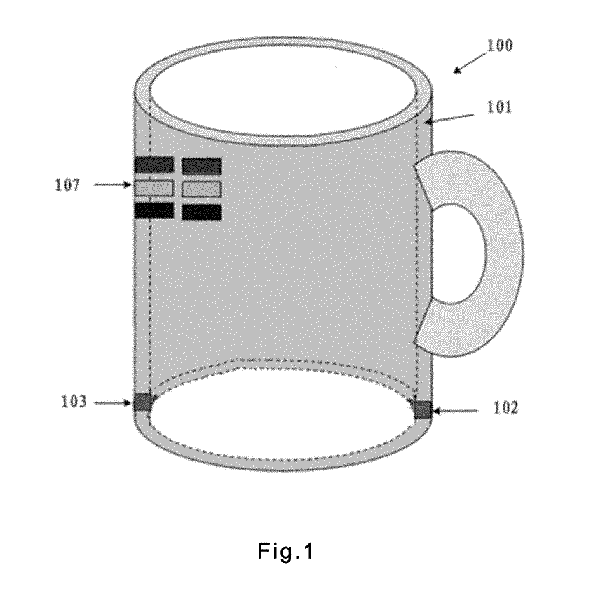

FIG. 1 is a schematic diagram of a tooth cup 100 according to an embodiment of the present disclosure. As shown in the figure, the tooth cup comprises a cup body 101. In the cup body 101, it may be integrated with, for example, a controller 102 and a purifier 103. The controller 102 may be configured to issue a purification command based on a predetermined condition. The purifier 103 may be configured to purify the tooth cup in response to receiving the purification command issued by the controller. Optionally, in the cup body 101, it may be further integrated with a display 107, as shown in FIG. 1. Additionally, in the cup body 101, it may be further integrated with any one or more of a power supply, a detector, a Bluetooth module, a timer, a memory, etc., which are some examples only and not shown in FIG. 1.

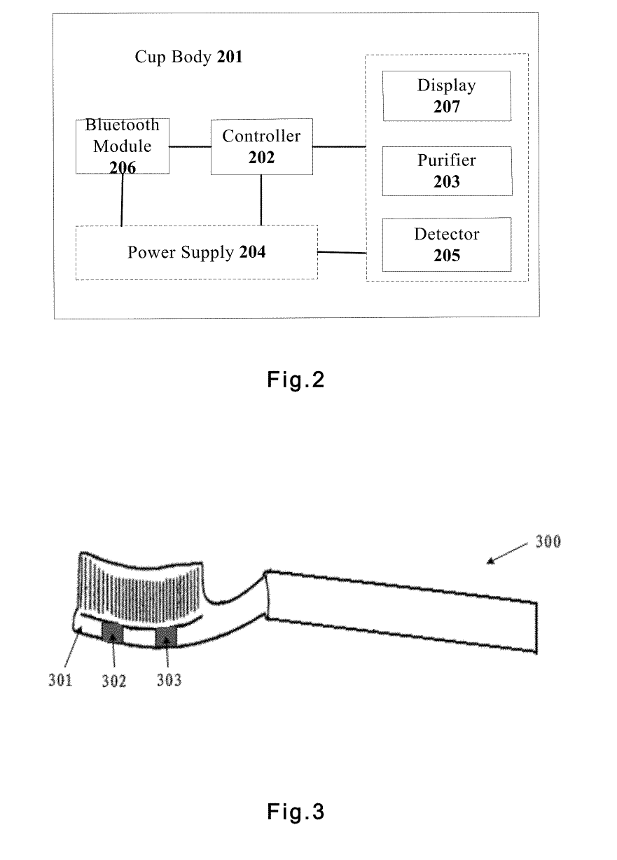

FIG. 2 is an exemplary structural diagram of a cup body 201 of a tooth cup according to an embodiment of the present disclosure. As shown in the figure, in the cup body, it is integrated with a controller 202 and a purifier 203. Additionally, in the cup body, it may be further integrated with one or more of a power supply 204, a detector 205, a Bluetooth module 206, and a display 207. Optionally, in the cup body, it may be further integrated with various types of sensors (not shown), e.g. a water volume sensor, a weight sensor, and so on.

The controller 202 may be configured to issue a purification command based on a predetermined condition. For example, the controller may be configured to obtain data from other integrated components in the tooth cup, and then analyze and process the obtained data. Then, the controller may determine the occurrence of the predetermined condition based on the analyzed and processed data, and performs actions based on this. The actions include, for example, issuing a purification command to the purifier. As an example, the controller may be configured to issue a purification command to the purifier based on the water volume within the tooth cup being reduced below a predetermined water volume threshold. The water volume within the tooth cup being reduced below a predetermined water volume threshold may be determined in many ways, e.g. by means of a water volume sensor or a weight sensor. The predetermined water volume threshold may be set by a user, e.g. to a value smaller than the water volume remained by the end of a brushing process conventionally. As an example, the controller may also issue a purification command to the purifier based on the water volume sensor sensing that the water volume in the tooth cup reduces from above a predetermined water volume threshold to below the predetermined water volume threshold. The controller may be implemented in a hardware unit and/or a software module, which hardware unit comprises an integrated circuit or a component of system-on-chip, an Application Specific Integrated Circuit (ASIC), a Field Programmable Gate Array (FPGA), a Complex Programmable Logic Device (CPLD), and so on.

The purifier 203 is configured to purify the tooth cup in response to receiving the purification command issued by the controller. The purifier comprises a microorganism purifier. The microorganism purifier may be e.g. a drier for drying the microorganism environment in the tooth cup. Alternatively or additionally, the purifier may also be a UV sterilizer for sterilizing the microorganism environment in the tooth cup. It should be noted that any unit or component capable of purifying the intra-cup environment is contemplated within the scope of the present disclosure.

The power supply 204 may be connected to the controller and the purifier and configured to supply power to the controller and/or the purifier. The power supply 204 may be also be configured to provide the power required for the operations of the other components (such as the controller, the purifier, the Bluetooth module, the display, etc.) in the cup body 201. The power supply may be in the form of a mains supply, or in the form of a battery. Optionally, the power supply may be a rechargeable battery.

The detector 205 may be configured to detect a degree of cleanness in the tooth cup. As an example, the detector may use a testing probe to detect the content of microorganism in the tooth cup. The testing probe may comprise luciferin-luciferase thereon and may be configured to capture and detect a luminous value of a photon generated from an interaction between the luciferin-luciferase and the remaining liquid in the tooth cup. By detecting the luminous value, the content of microorganism in the tooth cup can be acquired. It should be noted that other techniques or devices for detecting the degree of cleanness in the tooth cup are also contemplated within the scope of the present disclosure. As an example, the controller 202 may obtain data from the detector 205 and may analyze and process the data. Then, the controller issues a purification command to the purifier when analyzing that the degree of cleanness as detected by the detector is below a cleanness threshold. Additionally or alternatively, the user may directly issue to the purifier the purification command to purify the tooth cup, which can be implemented by e.g. a user directly pressing the purification switch button.

A Bluetooth module 206 may also be integrated in the cup body. The controller may communicate with an external device, especially an external mobile device (e.g. a mobile phone), by way of the Bluetooth module 206. For example, the controller 202 may forward the information about the degree of cleanness in the tooth cup to the external device through the Bluetooth module, in order that the user can know the degree of cleanness of the tooth cup at any time. For instance, the user may choose to brush his/her tooth when the degree of cleanness is relatively high. Alternatively or additionally, the controller 202 may receive data from the external device through the Bluetooth module, such as time data, image data (e.g. including video data), and even audio data, etc.

The display 207 may be connected to the controller. The display may be configured to display data under the control of the controller. The display may be, e.g. an LCD, an LED display, etc. The displayed data may be a degree of cleanness, time data, image data (e.g. including video data), and even audio data, etc., as described above.

FIG. 3 is a schematic diagram of a tooth brush 300 according to an aspect of the present disclosure. As shown in the figure, the tooth brush 300 comprises a brush head 301. In the tooth brush, it is integrated with a controller 302 and a purifier 303. Optionally, the tooth brush may also be integrated with a power supply for supplying power to the controller and/or the purifier. It should be noted that one or more of the detector, the Bluetooth module, the display and the sensor, as described above with reference to FIG. 2, may also be integrated in the tooth brush and is/are not shown in FIG. 3 in order to avoid repetition.

The controller 302 may be configured to issue a purification command based on a predetermined condition. The purifier 303 may be configured to purify the brush head in response to receiving the purification command issued by the controller. As described above with reference to FIG. 2, the purifier may comprise a microorganism purifier. The microorganism purifier may be a drier and/or a UV sterilizer, for example. Any unit or component capable of purifying the brush head is contemplated within the scope of the present disclosure.

As an example, in the tooth brush, it may be further integrated with a detector, as described above, for detecting a degree of cleanness of the brush head. As described with reference to FIG. 2, the controller may be configured to issue a purification command to the purifier based on the water volume on the brush head being reduced below a predetermined water volume threshold. The water volume on the brush head being reduced below a predetermined water volume threshold may be determined e.g. by means of a water volume sensor or a weight sensor. Alternatively or additionally, the controller is configured to issue a purification command when analyzing that the degree of cleanness as detected by the detector is below a cleanness threshold. Alternatively or additionally, the user may directly issue to the purifier the purification command to purify the brush head.

As another example, in the tooth brush, it may be further integrated with a Bluetooth module, and the controller may communicate with an external device through the Bluetooth module. Optionally, in the tooth brush, it may be further integrated with a display that may be connected to the controller. The display may be configured to display data under the control of the controller.

It should be noted that the units or components and their operational principles described above with reference to FIG. 2 may be applied alike in the tooth brush described herein. As described above, the controller may also issue a purification command to the purifier when the water volume sensor senses that the water volume on the brush head reduces from above a predetermined water volume threshold to below the predetermined water volume threshold. The detector may also detect the content of microorganism on the brush head using the testing probe as described with reference to FIG. 2. Optionally, the detector and/or purifier may be arranged in the brush head, in order to obtain a more accurate detection result and/or purification effect.

It should be understood that for clarity, the embodiments of the present disclosure are described with reference to different functional units or components. However, it will be apparent that the functionality of each functional unit may be implemented in a single unit, in a plurality of units or as a part of other functional units, without departing from the present disclosure. For example, the functionality described to be executed by a single unit may be executed by a plurality of different units. Therefore, the references to a particular functional unit are only regarded as references to an appropriate unit for providing the described functionality, rather than indicative of a strict logic or physical structure or organization. Therefore, the present disclosure may be implemented in a single unit, or physically and functionally distributed between different units and circuits.

Although the present disclosure has been described in conjunction with some embodiments, it is not intended to be limited to the specific forms illustrated in this context. Rather, the scope of the present disclosure is only limited by the appended claims. Additionally, although individual features may be included in different claims, these may possibly be advantageously combined, and the inclusion in different claims does not imply that a combination of features is not feasible and/or advantageous. The order of features in the claims does not imply any specific order in which the features must be worked. In addition, in the claims, the word “comprise” or “include” does not exclude other elements, and the indefinite article “a” or “an” does not exclude a plurality. Reference signs in the claims are provided merely as a clarifying example and shall not be construed as limiting the scope of the claims in any way.

1. A tooth cup comprising a cup body in which it is integrated with:
a controller configured to issue a purification command based on a predetermined condition; and
a purifier configured to purify the tooth cup in response to receiving the purification command issued by the controller.

2. The tooth cup according to claim 1, wherein in the cup body, it is further integrated with a power supply configured to supply power to at least one of the controller and the purifier.

3. The tooth cup according to claim 1, wherein the purifier comprises at least one of a drier and a UV sterilizer to purify the microorganism in the tooth cup.

4. The tooth cup according to claim 1, wherein in the cup body, it is further integrated with a detector configured to detect a degree of cleanness in the tooth cup.

5. The tooth cup according to claim 4, wherein the predetermined condition comprises the degree of cleanness as detected by the detector is below a cleanness threshold.

6. The tooth cup according to claim 4, wherein the detector comprises a testing probe comprising luciferin-luciferase thereon and configured to capture and detect a luminous value of a photon generated from an interaction between the luciferin-luciferase and the remaining liquid in the tooth cup.

7. The tooth cup according to claim 1, wherein the predetermined condition comprises the water volume in the tooth cup is reduced below a predetermined water volume threshold.

8. The tooth cup according to claim 1, wherein the purifier is further configured to purify the tooth cup in response to receiving a purification command issued by a user.

9. The tooth cup according to claim 1, wherein in the cup body, it is further integrated with a Bluetooth module through which the controller communicates with an external device.

10. The tooth cup according to claim 1, wherein in the cup body, it is further integrated with a display configured to display data under the control of the controller.

11. A tooth brush comprising a brush head in which it is integrated with:
a controller configured to issue a purification command based on a predetermined condition; and
a purifier configured to purify the brush head in response to receiving the purification command issued by the controller.

12. The tooth brush according to claim 11, wherein in the tooth brush, it is further integrated with a power supply configured to supply power to at least one of the controller and the purifier.

13. The tooth brush according to claim 11, wherein the purifier comprises at least one of a drier and a UV sterilizer to purify the microorganism on the brush head.

14. The tooth brush according to claim 11, wherein in the tooth brush, it is further integrated with a detector configured to detect a degree of cleanness of the brush head.

15. The tooth brush according to claim 14, wherein the predetermined condition comprises the degree of cleanness as detected by the detector is below a cleanness threshold.

16. The tooth brush according to claim 11, wherein the predetermined condition comprises the water volume on the brush head is reduced below a predetermined water volume threshold.

17. The tooth brush according to claim 11, wherein the purifier is further configured to purify the brush head in response to receiving a purification command issued by a user.

18. The tooth brush according to claim 11, wherein in the tooth brush, it is further integrated with a Bluetooth module through which the controller communicates with an external device.

19. The tooth brush according to claim 11, wherein in the tooth brush, it is further integrated with a display configured to display data under the control of the controller.

20. An oral cavity care system comprising at least one of the tooth cup according to claim 1 and a tooth brush, the tooth brush comprising a brush head in which it is integrated with:
a controller configured to issue a purification command based on a predetermined condition; and
a purifier configured to purify the brush head in response to receiving the purification command issued by the controller.
